(19) United States
(12) Patent Application Publication
(10) Pub. No.: US 2024/0260821 A1
(43) Pub. Date: Aug. 8, 2024

(54) FLUID SUPPLY DEVICES, ASSEMBLIES, AND METHODS FOR ENDOSCOPE SYSTEMS

(71) Applicant: Boston Scientific Scimed, Inc., Maple Grove, MN (US)

(72) Inventors: Ryan V. Wales, Northborough, MA (US); Ryan Vincent William Pollock, Sterling, MA (US); Rachel Perry, Lowell, MA (US); Brittany Elizabeth Reed, Brighton, MA (US)

(73) Assignee: Boston Scientific Scimed, Inc., Maple Grove, MN (US)

(21) Appl. No.: 18/430,235

(22) Filed: Feb. 1, 2024

Related U.S. Application Data
(60) Provisional application No. 63/442,957, filed on Feb. 2, 2023.

(51) Int. Cl. A61B 1/015; A61B 1/00; A61B 1/12

(52) U.S. Cl. CPC A61B 1/015; A61B 1/00119; A61B 1/00128; A61B 1/00137; A61B 1/126

(57) ABSTRACT

Devices, systems, and methods for providing liquid to an endoscope system. A fluid supply system may include a plurality of water reservoirs in fluid communication with one another to ensure a sufficient amount of water is available for endoscope procedures. The fluid supply system may include a first tubing subset configured to provide liquid to the endoscope and a second tubing subset configured to provide supplemental water to a reservoir in communication with the first tubing subset. Supplemental water may be pressurized via a pump, a liquid flow, and/or a via a gas flow. In some cases, a venturi siphon or a bridge siphon may be utilized to maintain pressure in the water reservoirs.

### CROSS-REFERENCE TO RELATED APPLICATIONS

This application claims the benefit of U.S. Provisional Patent Application Ser. No. 63/442,957 filed on Feb. 2, 2023, the disclosure of which is incorporated herein by reference.

### TECHNICAL FIELD

This disclosure relates generally to fluid supply devices, assemblies and methods, and particularly to fluid supply devices, assemblies, and methods for an endoscope system.

### BACKGROUND

A wide variety of intracorporeal and extracorporeal medical devices and systems have been developed for medical use, for example, for endoscopic procedures. Some of these devices and systems include guidewires, catheters, catheter systems, endoscopic instruments, and the like. These devices and systems are manufactured by any one of a variety of different manufacturing methods and may be used according to any one of a variety of methods. Of the known medical devices, systems, and methods, each has certain advantages and disadvantages. There is an ongoing need to provide alternative medical devices and systems as well as alternative methods for manufacturing and using medical devices and systems.

### SUMMARY

This disclosure provides design, material, manufacturing method, and use alternatives for medical devices and medical systems. In a first example, a fluid supply system for an endoscope may include a reservoir defining a volume, a first port in fluid communication with the volume and defining a first lumen and a first connector, a second port in fluid communication with the volume and having a second lumen and a second connector, a first tubing subset having a tube with a first end configured to couple to the first port and a second end configured to couple to an endoscope component, and a second tubing subset having a tube with a first end configured to couple to the second port and a second end configured to couple to a first port in fluid communication with a volume of an additional reservoir.

Alternatively or additionally to any of the examples above, in another example, the fluid supply system may include a cap configured to couple to the reservoir, and the cap may include the first port and the second port.

Alternatively or additionally to any of the examples above, in another example, the first end of the tube of the first tubing subset may be configured to be permanently secured to the first port.

Alternatively or additionally to any of the examples above, in another example, the second end of the tube of the first tubing subset may be configured to couple to an endoscope includes an endoscope connector having a water port and a gas port, and the second end of the tube of the second tubing subset may be configured to couple to the port in fluid communication with a volume of the additional reservoir includes an endoscope connector having a water port and a gas port.

Alternatively or additionally to any of the examples above, in another example, the second tubing subset comprises a first fluid path in communication with a gas source and a second fluid path in communication with the volume of the additional reservoir.

Alternatively or additionally to any of the examples above, in another example, the second tubing subset may comprise a connector coupling a first length of tubing including the first fluid path and a second length of tubing including the second fluid path with a third length of tubing including the first fluid path and the second fluid path.

Alternatively or additionally to any of the examples above, in another example, the first tubing subset may be in fluid communication with a pump to pump fluid out of the volume of the reservoir to the endoscope.

Alternatively or additionally to any of the examples above, in another example, the first tubing subset may include a first length of tubing extending from the reservoir to the pump and a second length of tubing from the pump to a valve, the valve may be in fluid communication with tubing configured to extend to the endoscope and the second tubing subset, and the valve may be configured to proportion a portion of the fluid received at the valve to the tubing configured to extend to the endoscope and a portion of the fluid received at the valve to the second tubing subset.

Alternatively or additionally to any of the examples above, in another example, the second tubing subset may comprise a connector, a first length of tubing in fluid communication with the valve, a second length of tubing configured to be in fluid communication with the volume of the additional reservoir, and a third length of tubing in fluid communication with the reservoir and configured to receive fluid from the first length of tubing and the second length of tubing, and wherein the connector couples the first length of tubing and the second length of tubing with the third length of tubing.

Alternatively or additionally to any of the examples above, in another example, the reservoir may be a first reservoir of a plurality of reservoirs and the plurality of reservoirs include a last reservoir, the first tubing subset may be configured to supply water from the first reservoir to a water port of an endoscope connector at the second end of the tube of the first tubing subset, the second end of tube of the second tubing subset may be configured to be in fluid communication with water from the last reservoir, and a second tube of the second tubing subset may have a first end configured to couple to a first port in communication with a volume of the last reservoir and a second end configured to be in fluid communication with a gas port on the endoscope connector.

In another example, a tubing set for use with an endoscope system may include a first length of tubing having a first end and a second end, an endoscope connector including a gas port and a water port, the water port is fluidly coupled to the first end of the first length of tubing, and a manifold system configured to fluidly couple a plurality of reservoirs to provide water from the plurality of reservoirs to a water port of the endoscope connector.

Alternatively or additionally to any of the examples above, in another example, the manifold system is incorporated in a cap configured to couple to the plurality of reservoirs and be in fluid communication with the second end of the first length of tubing.

Alternatively or additionally to any of the examples above, in another example, the cap includes a plurality of sets of threads, each set of threads is configured to couple to a reservoir of the plurality of reservoirs.

Alternatively or additionally to any of the examples above, in another example, the manifold system comprises a second length of tubing configured to fluidly couple a second reservoir to a first reservoir of the plurality of reservoirs, the first reservoir is in fluid communication with the second end of the first length of tubing, a third length of tubing configured to fluidly couple a third reservoir of the plurality of reservoirs to the second reservoir, and a fourth length of tubing coupled to the gas port of the endoscope connector and configured to fluidly couple the gas port to the third reservoir.

Alternatively or additionally to any of the examples above, in another example, the manifold system may include a second length of tubing configured to be in fluid communication with a first reservoir of the plurality of reservoirs, a third length of tubing configured to be in fluid communication with a second reservoir of the plurality of reservoirs, a fourth length of tubing configured to be in fluid communication with a fluid source, and a connector coupling a second length of tubing, the third length of tubing, and the fourth length of tubing, the connector is configured to cause fluid from the second reservoir to travel through the third length of tubing to the second length of tubing in response to the manifold receiving fluid from the fourth length of tubing.

Alternatively or additionally to any of the examples above, in another example, the fluid source is a gas source.

Alternatively or additionally to any of the examples above, in another example, the fourth length of tubing is configured to fluidly couple to a gas port of the endoscope connector and the fluid source is a source of gas provided to the endoscope connector.

Alternatively or additionally to any of the examples above, in another example, the fluid source is a water source.

In another example, a fluid supply system for an endoscope, may include a plurality of water reservoirs, a manifold system in communication with the plurality of water reservoirs, and an endoscope connector configured to connect to an endoscope and in fluid communication with at least one of the plurality of water reservoirs, and wherein the manifold system is configured to provide water from the plurality of reservoirs through the endoscope connector in response to a call for water from the endoscope.

Alternatively or additionally to any of the examples above, in another example, a volume of the plurality of reservoirs and a fluid path through the manifold system may be pressurized in response to the call for water from the endoscope.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various embodiments and together with the description serve to explain the principles of the present disclosure.

FIG. 1 depicts a schematic view of components of an illustrative endoscope;

FIG. 2 depicts a schematic view of components of an illustrative endoscope system;

FIG. 3A depicts a schematic view of an illustrative endoscope system, wherein the endoscope system is activated to deliver air to atmosphere;

FIG. 3B depicts a schematic view of an illustrative endoscope system, wherein the endoscope system is activated to deliver air to a patient through the patient end of the endoscope;

FIG. 3C depicts a schematic view of an illustrative endoscope system, wherein the endoscope system is activated to deliver lens wash fluid through the patient end of the endoscope;

FIG. 3D depicts a schematic view of an illustrative endoscope system, wherein the endoscope system is activated to deliver irrigation fluid through the patient end of the endoscope;

FIG. 4 depicts a schematic view of an illustrative hybrid endoscope system;

FIG. 5 depicts a schematic cross-section view of ports for coupling with tubing;

FIG. 6 depicts a schematic side of view of an illustrative fluid supply system;

FIG. 7 depicts a schematic view of an illustrative fluid supply system;

FIG. 8 depicts a schematic view of an illustrative fluid supply system;

FIG. 9 depicts a schematic view of an illustrative fluid supply system;

FIG. 10 depicts a schematic side view of an illustrative fluid supply system; and

FIG. 11 depicts a schematic bottom view of an illustrative manifold system of the fluid supply system depicted in FIG. 10.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

This disclosure is now described with reference to an illustrative medical system that may be used in endoscopic medical procedures. However, it should be noted that reference to this particular procedure is provided only for convenience and not intended to limit the disclosure. A person of ordinary skill in the art would recognize that the concepts underlying the disclosed devices, systems, assemblies, and/or related methods of use may be utilized in any suitable procedure, medical or otherwise. This disclosure may be understood with reference to the following description and the appended drawings, wherein like elements are referred to with the same reference numerals.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features, and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise.

As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is illustrative only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The detailed description is intended to illustrate but not limit the disclosure. Those skilled in the art will recognize that the various elements described may be arranged in various combinations and configurations without departing from the scope of the disclosure. The detailed description illustrates example embodiments of the disclosure.

An endoscope is used in performing diagnostic and/or therapeutic treatments by inserting an elongated shaft of the endoscope into a subject to observe a part to be examined within a body cavity of the subject and, if necessary, inserting a treatment instrument/tool into a working channel in the elongated shaft of the endoscope. Such endoscopes or endoscope systems may include a fluid/lens wash capability, or the like, configured to feed fluid, such as gas (e.g., air, CO₂), to an end of the endoscope for insufflating the inside of the subject at a target site. Lens wash features may provide sterilized water at relatively high pressure to spray across and clear debris from a camera lens of the endoscope. In order to rinse the target site of the subject, separate from the air/water feed capability, endoscopes or endoscope systems may have an irrigation capability that provides lower pressure, higher volume water, supplied via a pump (e.g., a peristaltic pump) to the target site in order to clear the field of view for observation and treatment. A fluid supply system for an endoscope may have a water source (e.g., a fluid source) for lens wash and/or irrigation features that may include one or more fluid reservoirs having tubing and fitting (e.g., caps, covers, ports, etc.) assemblies that create a plumbing circuit in connection with the endoscope channels, valving, and/or connectors to accomplish the gas and water functions described.

Tubing and fitting assemblies may be available in various configurations, which may include or be configured to be in communication with a reservoir or other suitable fluid source (e.g., one or more water bottles, etc.), a fitting fitted for the specific bottle, and an array of tubing (e.g., one or more tubing sets) that is extendable through openings and/or configured to connect to connectors or ports in the fitting or other portions of a water reservoir. The tubing may be arranged to accommodate a specific configuration of endoscope fittings and valving, which does not tend to be modular or optional. In some cases, one or more connectors or ports may be utilized to connect tubing for irrigation, lens wash, and/or insufflation features to an endoscope umbilical in fluid communication with working channels of the endoscope.

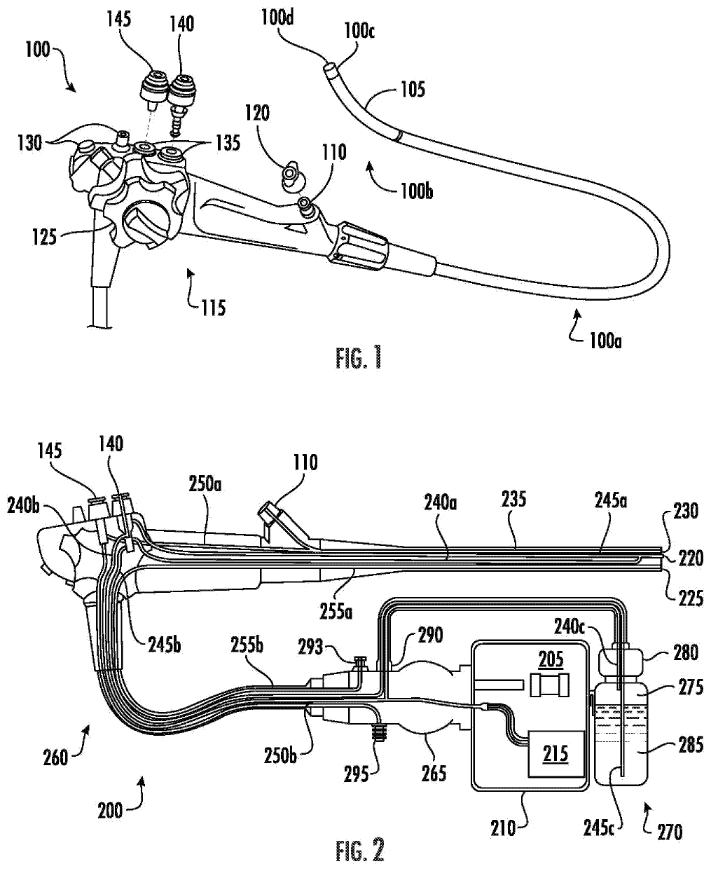

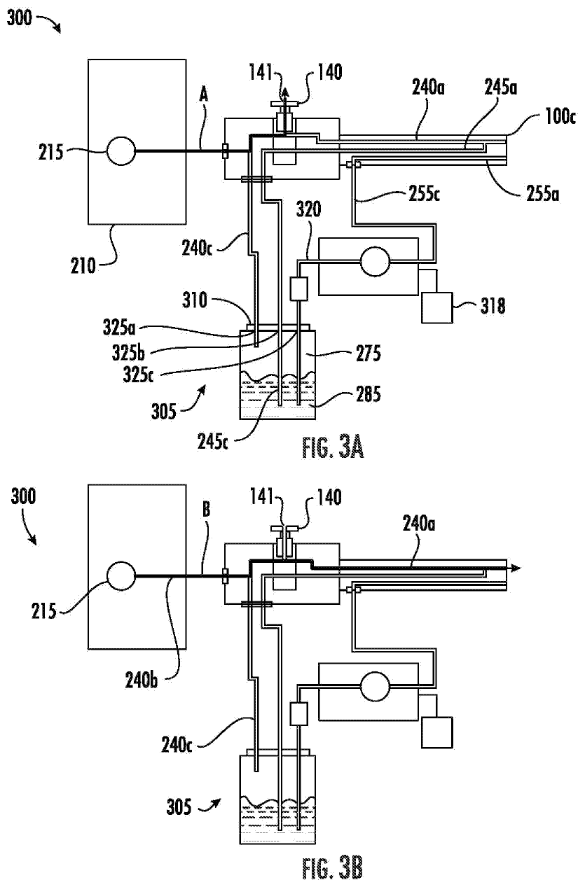

With reference to FIG. 1, an illustrative endoscope 100 is depicted and FIG. 2 depicts an illustrative endoscope system 200. The endoscope 100 may include an elongated tube or shaft 100a that is configured to be inserted into a subject (e.g., a patient). Details of the endoscope 100 and the endoscope system 200 may be more fully described in U.S. patent Application Publication No. 2022/0192479 A1, filed on Dec. 21, 2021, and titled TUBING ASSEMBLIES AND METHODS FOR FLUID DELIVERY, which is hereby incorporated by reference in its entirety for all purposes.

A light source 205 of the endoscope system 200 may feed illumination light to a distal portion 100b of the endoscope 100. The distal portion 100b of the endoscope 100 may house an imager (e.g., CCD or CMOS imager) (not shown). The light source 205 (e.g., a lamp) may be located in a video processing unit 210 that processes signals input from the imager and outputs processed video signals to a video monitor (not shown) for viewing. The video processing unit 210 may also serve as a component of an air/water feed circuit by housing a pressurizing or air pump 215, such as an air feed pump, in the unit 210. Other suitable pumps for the air/water feed circuit are contemplated.

The endoscope shaft 100a may include a distal tip 100c (e.g., a distal tip unit) provided at the distal portion 100b of the shaft 100a and a flexible bending portion 105 proximal to the distal tip 100c. The flexible bending portion 105 may include an articulation joint (not shown) to assist with steering the distal tip 100c. On an end face 100d of the distal tip 100c of the endoscope 100 is a gas/lens wash nozzle 220 for supplying gas to insufflate the interior of the subject at the treatment area and for supplying water to wash a lens covering the imager. An irrigation opening 225 in the end face 100d supplies irrigation fluid to the treatment area of the subject. Illumination windows (not shown) that convey illumination light to the treatment area, and an opening 230 to a working channel 235 extending along the shaft 100a for passing tools to the treatment area, may also be included on the face 100d of the distal tip 100c. The working channel 235 may extend along the shaft 100a to a proximal channel opening 110 positioned distal to an operating handle 115 (e.g., a proximal handle) of the endoscope 100. A biopsy valve 120 may be utilized to seal the channel opening 110 against unwanted fluid egress.

The operating handle 115 may be provided with knobs 125 for providing remote 4-way steering of the distal tip via wires connected to the articulation joint in the flexible bending portion 105 (e.g., one knob may control up-down steering and another knob may control left-right steering). A plurality of video switches 130 for remotely operating the video processing unit 210 may be arranged on a proximal end side of the operating handle 115.

The operating handle 115 may be provided with dual valve locations 135. One of the valve locations 135 (e.g., locations at valve wells) may receive a gas/water valve 140 for operating an insufflating gas and lens water feed operation. A gas supply line 240a and a lens wash line 245a run distally from the gas/water valve 140 along the shaft 100a and converge at the distal tip 100c proximal to the gas/wash nozzle 220 (FIG. 2).

The other valve location 135 may receive a suction valve 145 for operating a suction operation. A suction supply line 250a may run distally from the suction valve 145 along the shaft 100a to a junction point in fluid communication with the working channel 235 of the endoscope 100.

The operating handle 115 may be electrically and fluidly connected to the video processing unit 210, via a flexible umbilical 260 and connector portion 265 extending therebetween. The flexible umbilical 260 may have a gas (e.g., air or CO₂) feed line 240b, a lens wash feed line 245b, a suction feed line 250b, an irrigation feed line 255b, a light guide (not shown), an electrical signal cable (not shown), and/or other suitable lines, guides, and/or cables. The connector portion 265 when plugged into the video processing unit 210 connects the light source 205 in the video processing unit with the light guide. The light guide may run along the umbilical 260 and the length of the endoscope shaft 100a to transmit light to the distal tip 100c of the endoscope 100. The connector portion 265, when plugged into the video processing unit 210, may also connect the air pump 215 to the gas feed line 240b in the umbilical 260.

A fluid source, such as a fluid or water container or reservoir 270 (e.g., water bottle, bag, etc.), a building water source, and/or other suitable fluid source, may be fluidly connected to the endoscope 100 through the connector portion 265 and the umbilical 260. A length of gas supply tubing 240c may pass from one end positioned in an air gap or gas 275 between the top 280 (e.g., a bottle cap, lid, closure, cover, etc.) of the reservoir 270 and the remaining water 285 (e.g., the remaining water 285) in the reservoir to a connector 290 on the outside of the connector portion 265. The gas feed line 240b from the umbilical 260 branches in the connector portion 265 to fluidly communicate with the gas supply tubing 240c at the detachable connector 290, as well as the air pump 215. A length of lens wash tubing 245c, with one end positioned at the bottom of the reservoir 270, may pass through the top 280 of the reservoir 270 to the same detachable connector 290 as the gas supply tubing 240c on the connector portion 265. In other embodiments, the connections may be separate and/or separated from each other. The connector portion 265 may also have a detachable irrigation connection 293 for irrigation supply tubing (not shown) running from a source of irrigation water (not shown) to the irrigation feed line 255b in the umbilical 260. In some configurations, irrigation water may be supplied via a pump (e.g., peristaltic pump) from a water source (not shown) independent from the water reservoir 270. In other embodiments, the irrigation supply tubing and lens wash tubing 245c may source water from the same reservoir. The connector portion 265 may also include a detachable suction connection 295 for suction feed line 250b and suction supply line 250a fluidly connecting a vacuum source (e.g., hospital house suction) (not shown) to the umbilical 260 and endoscope 100.

The gas feed line 240b and lens wash feed line 245b may be fluidly connected to the valve location 135 for the gas/water valve 140 and configured such that operation of the gas/water valve 140 in the well controls supply of gas or lens wash to the distal tip 100c of the endoscope 100. The suction feed line 250b is fluidly connected to the valve location 135 for the suction valve 145 and configured such that operation of the suction valve 145 in the well controls suction applied to the working channel 235 of the endoscope 100.

The gas supply tubing 240c and the lens wash tubing 245c may be combined in a coaxial relationship, but this is not required. In one example, the gas supply tubing 240c may define a lumen that is sufficiently large in diameter to encompass a smaller diameter lens wash tubing 245c, coaxially received within the gas supply tubing, as well as provide air to the water source in an annular space surrounding the lens wash tubing to pressurize the water reservoir. The lens wash tubing 245c may be configured to exit the lumen defined by the coaxial gas supply tubing in any suitable sealed manner, such as for example via an aperture, fitting, collar, and/or the link for the purpose of transitioning from the coaxial arrangement to a side-by-side arrangement of the detachable gas/lens wash connection to the endoscope connector portion 265.

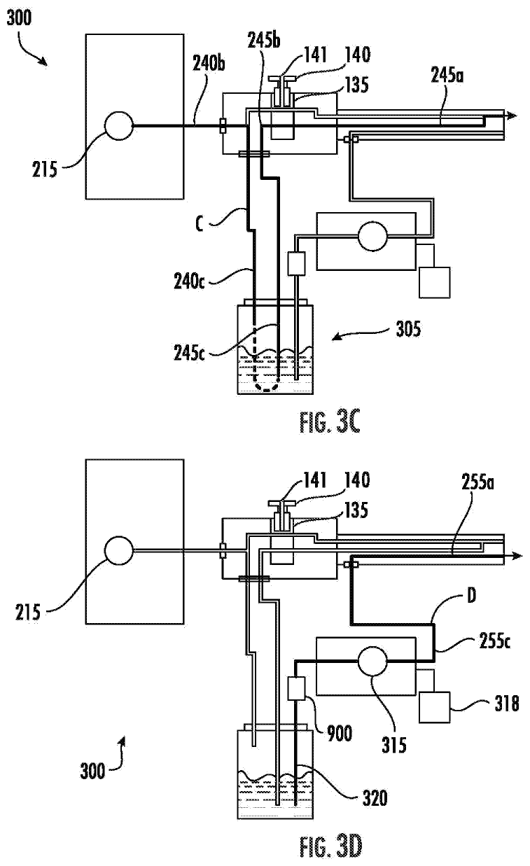

FIGS. 3A-3D are schematic views illustrating an operation of an embodiment of an endoscope system 300 (e.g., a medical device assembly), which may be similar or dissimilar to the endoscope system 200, where the supply tubing for irrigation and lens wash are connected to and drawn from a single water reservoir 270, 305 and/or other suitable fluid source. The hybrid system 300 may include the one or more water reservoir 270, 305 (e.g., a single water reservoir 305, as depicted in FIGS. 3A-3D), a cover or cap 310 (e.g., a fitting) to cover an opening of the reservoir 270, 305, gas supply tubing 240c, lens wash tubing 245c, irrigation pump 315 that may be in communication with foot switch 318 or other suitable switch, upstream irrigation tubing 255c, 320, and downstream irrigation supply tubing 255c.

The cap 310 may be configured to attach in a seal-tight manner (e.g., a hermetic seal or other suitable seal) to the water reservoir 270, 305 by a threaded arrangement and/or other suitable coupling mechanism. The cap 310 may include a gasket to seal the cap 310 to the reservoir 270, 305. The gasket may be an O-ring, flange, collar, and/or the like and can be formed of any suitable material. A number of through-openings (325a, 325b, 325c) in the cap 310 may be provided to receive, respectively, a length of gas supply tubing 240c, a length of lens wash tubing 245c, and a length of upstream irrigation supply tubing 320, but this is not required. Alternatively or additionally, the through-openings may be positioned at other locations on the reservoir, optionally independent of the cap 310. In FIGS. 3A-3D, the system depicted includes separate tubing for gas supply, lens wash, and irrigation.

In other embodiments, the gas supply tubing 240c and lens wash tubing 245c may be combined in a coaxial arrangement. For example, the gas supply tubing may define a lumen that is sufficiently large in diameter to encompass a smaller diameter lens wash tubing, coaxially received within the gas supply tubing, as well as provide air to the water source in an annular space surrounding the lens wash tubing to pressurize the water reservoir. The lens wash tubing 245c may be configured to exit the lumen defined by the coaxial gas supply tubing in any suitable sealed manner, such as, for example, an aperture, fitting, collar, and/or the like, for the purpose of transitioning from the coaxial arrangement to a side-by-side arrangement at the detachable gas/lens wash connection to the endoscope connector portion 265 (e.g., FIG. 2).

In various embodiments, different configurations of valving may be incorporated into the tubing of the system 200, 300. For example, an in-flow check valve may be disposed in the path of the gas supply tubing 240c to help prevent liquid backflow into the air pump 215. In this manner, pressure building within the water reservoir 270, 305 may create a pressure difference between the water reservoir 270, 305 and the gas supply tubing 240c helping to maintain a positive pressure in the water reservoir 270, 305 even when large amounts of water may be removed from the water source during the irrigation function. This arrangement may compensate for any time lag in air being delivered from the air pump 215 to the water reservoir 270, 305, which might otherwise cause a negative pressure vacuum in the water reservoir 270, 305. Similarly, an out-flow check valve, such as a one-way valve, may be incorporated in the lens wash tubing 245c, upstream irrigation supply tubing 320, and/or downstream irrigation supply tubing 255c to help prevent backflow of water from either or both of the lens wash and irrigation tubing in the event of a negative pressure situation, as described.

More generally, in some configurations, a check valve may refer to any type of configuration for fluid to flow only in one direction in a passive manner. For example, a check valve may include, or refer to, one or more of a ball check valve, a diaphragm check valve, a swing check valve, a tilting disc check valve, a flapper valve, a stop-check valve, a lift-check valve, an in-line check valve, a duckbill valve, a pneumatic non-return valve, a reed valve, a flow check, a flapper valve, and/or other suitable check valve. Accordingly, a check valve as used herein may be meant to be separate and distinct from an active valve that is operated in a binary manner as an on/off valve or switch to allow flow to be turned on or allow flow to be turned off (e.g., a stop cock valve, solenoid valve, peristaltic pump, blow off valve).

During operation of the system of FIGS. 3A-3D, a flow of water for irrigation may be achieved by operating the irrigation pump 315 via the foot switch 318 and/or other suitable actuation mechanism. A flow of water for lens wash may be achieved by depressing the gas/water valve 140 on the operating handle 115 of the endoscope 100. These functions may be performed independent of one another or simultaneously. When operating lens wash and irrigation at the same time, as fluid is removed from the water reservoir 270, 305, the pressure in the system may be controlled to maintain the lens wash tubing 245c at substantially the pressure necessary to accomplish a lower flow rate lens wash, while compensating for reduced pressure in the water reservoir 270, 305 due to supplying a high flow rate irrigation. When pressure is reduced in the water reservoir 270, 305 by use of the lens wash function, the irrigation function, or both functions simultaneously, the reduced pressure may be compensated for by the air pump 215 via the gas supply tubing 240c.

Flow paths in the schematic set-ups depicted in each of FIGS. 3A-3D have been highlighted to show the different flow paths possible with the hybrid system 300 having supply tubing (e.g., irrigation tubing 255c, 320, lens wash tubing 245c, and/or other suitable tubing) connected to and drawn from the single water reservoir 270, 305. Not all features depicted in each of FIGS. 3A-3D are labeled with reference numerals in each of FIGS. 3A-3D for clarity purposes, but similarly depicted features in FIGS. 3A-3D should be understood to be referring to a same or similar feature in each of FIGS. 3A-3D.

As shown in FIG. 3A, the endoscope 100 may be in a neutral state with the gas/water valve 140 in an open position. The neutral state delivers neither gas, nor lens wash fluid, to the distal tip of the endoscope. Rather gas (pressure) is delivered along path A from the pressurizing air pump 215 and vented through the gas feed line 240b (e.g., in the umbilical 260 via the connector portion 265, as depicted in FIG. 2) and through the gas/water valve 140 to atmosphere. Because the system is open at the vent hole in the gas/water valve 140, there is no build up to pressurize the water reservoir 270, 305 and consequently no water is pushed through the lens wash tubing 245c.

As shown in FIG. 3B, the endoscope 100 may be in a gas delivery state with the gas/water valve 140 in a first position. When gas is called for at the distal tip 100c, for example, to clean the end face 100d of the distal tip 100c or insufflate the patient body in the treatment area, the user may close off a vent hole 141 in the gas/water valve 140 with a thumb, finger, or the like (first position). In this state, gas (pressure) may be delivered along path B from the air pump 215 and flowed through the gas feed line 240b (e.g., in the umbilical 260 via the connector portion 265, as depicted in FIG. 2). The gas may continue through the gas/water valve 140 to the gas supply line 240a in the endoscope shaft 100a and out the gas/lens wash nozzle 220 at the distal tip 100c. There is no build up to pressurize the water reservoir since the system is open at the gas/lens wash nozzle 220, and consequently no liquid is pushed through the lens wash tubing 245c.

As shown in FIG. 3C, the endoscope 100 may be in a lens wash delivery state with the gas/water valve 140 in a second position. When lens wash is called for at the distal tip 100c, for example, to clean the end face 100d of the distal tip 100c, the user, keeping the vent hole 141 in the gas/water valve 140 closed off, depresses the valve 140 to its furthest point in the valve location 135. The second position blocks off the gas supply to both atmosphere and the gas supply line 240a in the endoscope 100, and opens up the gas/water valve 140 to allow lens wash water to pass through to the lens wash line 245a in the endoscope shaft 100a and out the gas/lens wash nozzle 220 at the distal tip 100c. In this state, gas (pressure) is delivered along path C from the air pump 215, through the branched line in the connector portion 265 and out of the gas supply tubing 240c to the water reservoir 270, 305. The gas (pressure) pressurizes the surface of the remaining water 285 in the reservoir 270, 305 and pushes water up the lens wash tubing 245c (e.g., to the connector portion 265 of the umbilical 26, as depicted in FIG. 2. The pressurized lens wash water may be pushed further through the lens wash feed line 245b and through the gas/water valve 140. Because the system 300 is closed, gas pressure may be allowed to build and maintain a calibrated pressure level in the water reservoir 270, 305, rather than venting to atmosphere or being delivered to the patient. This pressure, along with the endoscope feed and supply lines and external tubing, translates to a certain range of flow rate of the lens wash.

As shown in FIG. 3D, the endoscope 100 is in an irrigation delivery state. This may be performed at the same or a different time from the delivery of gas and/or lens wash. When irrigation is called for at the distal tip 100c, for example, if visibility in the treatment area is poor or blocked by debris, or the like, the user may activate the irrigation pump 315 (e.g., by depressing foot switch 318 or other suitable actuation mechanism) to deliver water or other liquid from the reservoir 270, 305 along path D. With the pump 315 activated, water is sucked out of the water reservoir 270, 305 through the upstream irrigation supply tubing 255c, 320 and pumped along the downstream irrigation supply tubing 255c to the connector portion 265. The irrigation pump head pressure pushes the irrigation water further through the irrigation feed line 255b (e.g., extending through the umbilical 260), through the irrigation supply line 255a in the endoscope shaft 100a, and out the irrigation opening 225 at the distal tip 100c. The irrigation pump pressure may be calibrated, along with the endoscope irrigation feed and supply lines and external tubing, to deliver a certain range of flow rates of the irrigation fluid.

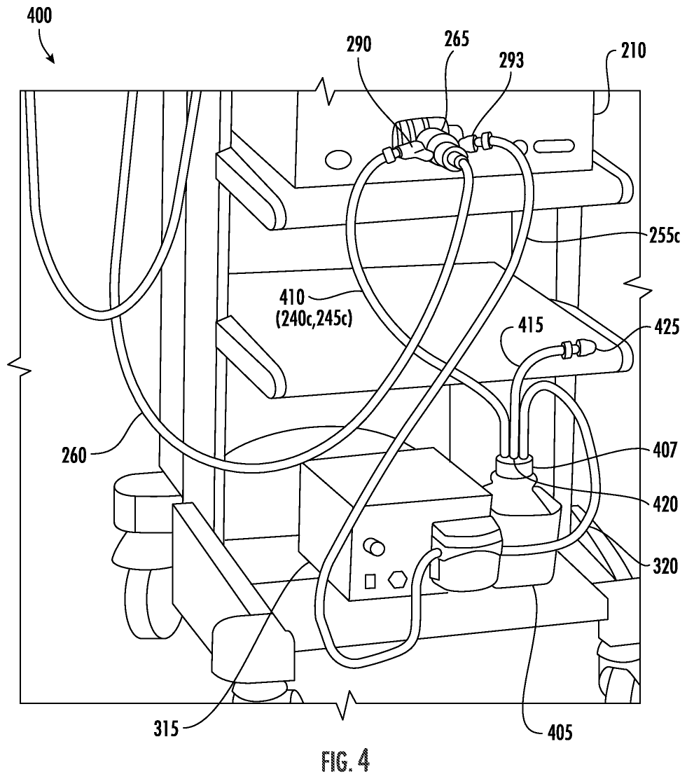

FIG. 4 is a schematic drawing illustrating a further embodiment of a hybrid system 400 including a video processing unit 210, connector portion 265, peristaltic irrigation pump 315, water reservoir 405 and top 407, coaxial gas and lens wash supply tubing 410, upstream and downstream irrigation supply tubing 320, 255c (e.g., first and second lengths of tubing), and alternative gas supply tubing 415 (e.g., CO₂). A length of the alternative gas supply tubing 415 passes from one end positioned in the gas gap 275 between the top 407 of the water reservoir 405 and the remaining water 285 in the reservoir through an additional opening 420 in the top 407 of the reservoir 405 to a detachable connection 425 for a source of the alternative gas supply (e.g., hospital house gas source of CO₂). When the alternative gas supply is desired, such as CO₂ gas, the air pump 215 on the video processing unit 210 may be turned off and CO₂ gas, rather than air, is thereby flowed to the water reservoir 405 pressurizing the water surface. In the neutral state, CO₂ gas flows backward up the gas supply tubing 240c to the connector portion 265, up the gas feed line 240b, and is vented through the gas/water valve 140 to atmosphere. In the first position, the user closes off the vent hole in the gas/water valve 140, and the CO₂ gas is flowed through the gas/water valve to the gas supply line 240a in the endoscope shaft 100a and out the gas/lens wash nozzle 220 at the distal tip 100c. In the second position, the user depresses the valve 140 to the bottom of the valve location 135, keeping the vent hole in the gas/water valve closed off. The second position blocks the CO₂ gas supply to both atmosphere and the gas supply line 240a in the endoscope 100, and opens up the gas/water valve 140 to allow lens wash water to pass through to the lens wash line 245a (e.g., a supply line) in the endoscope shaft 100a and out the gas/lens wash nozzle 220 at the distal tip 100c. Gas (pressure) in the reservoir 405 is maintained by delivery gas through alternative gas (e.g., CO₂) supply tubing 415. The irrigation function may be accomplished in a similar manner as the operation described above with respect to FIG. 3D.

One of the challenges faced during endoscopic procedures is that the common water reservoir and tube set used have a maximum capacity (e.g., a maximum of 1 liter of water or other set amount) and are not designed to be refilled. In some cases, physicians may utilize a combination of water irrigation and lens wash during endoscopic procedures (e.g., during both regular and complex procedures). A hybrid tubing set may be used to obtain sterile water from a single water reservoir of limited capacity for both irrigation and lens wash functions during an endoscope procedure. For a facility, such as a hospital or endoscopic outpatient facilitate, a daily load of procedures on an endoscope system may require a water volume equivalent to two, three, or more water reservoirs of limited size (e.g., one-liter sized reservoirs and/or other suitably sized reservoirs). This may force nurses/technicians to replace the water reservoir one or more times a day, which is an extra step over standard procedures, may delay subsequent procedures, may introduce multiple opportunities for contamination to the tube set by either contacting non-sterile surfaces or dropping the tubing on the floor, and/or may cause and/or result in one or more other issues.

Disclosed herein are methods and systems to reduce or eliminate the need to disconnect the tube set from a water reservoir and/or the endoscope system and to provide a sufficient water volume of water for a desired amount of procedures. In one example fluid supply system configured to supply a suitable volume of fluid, two or more water reservoirs may be fluidly coupled together to prevent the need to replace an initial or primary water reservoir and giving physicians access to any amount of fluid desired in increments (e.g., additional or fewer reservoirs may be provided depending on a number of procedures that are intended to be performed during a day, a number of add-on procedures during a day, a number of no-shows for procedures during a day, etc.)

To facilitate creating a fluid supply system in which two or more water reservoirs may be fluidly coupled to one another, a water reservoir, fitting, cover, and/or cap may be utilized that includes two or more ports configured to couple to a tubing set or tubing sub-sets. The two or more ports may include or be in fluid communication with holes, openings, and/or lumens leading to an interior volume of and/or defined by the water reservoir. Example ports may include a first port configured to engage with an air, water, or air/water tube sub-set that may be permanently or releasably coupled to the first port and a second port configured to be releasably or permanently coupled to a tube sub-set configured to couple to an additional reservoir, but additional and/or alternative suitable ports are contemplated. In some cases, one or more of the ports may include a shoulder feature on an outer face of the water reservoir, fitting, cover, and/or cap.

The components and/or configuration of the fluid supply systems discussed herein may be configured to provide supplemental water to a primary water reservoir and/or otherwise ensure a user of an endoscope has a sufficient volume of water. The fluid supply systems may be configured to provide water to the primary water reservoir and/or to an endoscope in response to a call for water from the endoscope by ensuring one or more supplemental water reservoirs and manifold systems or tubing subsets fluidly coupled to the primary water reservoir and/or the endoscope remain pressurized as fluid is removed from the primary water reservoir and/or otherwise provided to the endoscope.

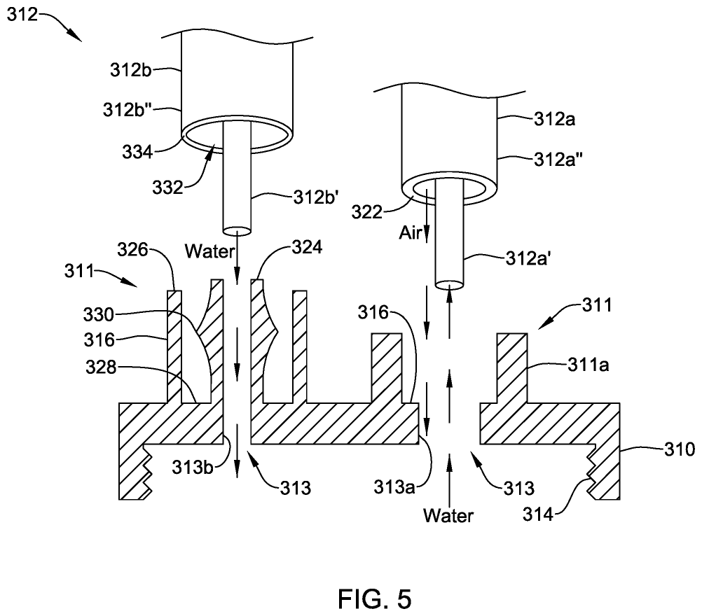

FIG. 5 depicts a schematic cross-section view of illustrative ports 311 on a fitting for a water reservoir (e.g., a cap 310 and/or other suitable fitting) and defining or in communication with holes 313 (e.g., lumens) configured to lead to an interior volume of a water reservoir, where the ports 311 are configured to couple with a tubing set 312 that facilitates providing an adequate supply of water to an endoscope. In some cases, when the fitting is the cap 310 or another suitable fitting, the fitting may be configured to be releasably coupled to a water reservoir in a hermetic manner. Example coupling techniques for coupling the fitting with the water reservoir may include, but are not limited to, a threaded connection via threads 314, a luer lock connection, a bayonet connection, a snap connection, a press fit connection, and/or one or more other suitable coupling techniques.

As depicted in FIG. 5, a first port 311a may include, define, and/or be in fluid communication with a first hole or opening 313a (e.g., a first lumen and/or other suitable hole or opening) and a second port 311b may include, define, and/or be in fluid communication with a second hole or opening 313b (e.g., a second lumen and/or other suitable hole or opening). The first port 311a be configured to couple with a first manifold system or tubing subset 312a (e.g., an air/water or lens wash tubing subset or manifold system) having a first end configured to couple with the first port 311a and a second end having a connector (e.g., the connector 290 and/or other suitable connector) configured to couple with an umbilical of an endoscope. The first port 311a may include a shoulder 316 on an outer surface of the cap 310 that is configured to receive an end surface 322 of first tubing subset 312a, where the first tubing subset 312a may be coaxial tubing with a water passage defined by an inner tube 312a′ for removing water from a water reservoir and a gas or air passage defined as the space between the inner tube 312a′ and an outer tube 312a″ for passing gas or air into the water reservoir. In addition to or as an alternative to the first port 311a being configured to couple to the first tubing subset 312a, the first port may be configured to couple with other tubing in communication with an endoscope system including, but not limited to, irrigation tubing subsets, refill tubing subsets, a connector in communication with multiple tubing subsets, and/or other suitable tubing or connectors thereof.

The first port 311a may be configured to couple with the first tubing subset 312a and/or other suitable tubing or tubing subsets of the tubing set 312 in any suitable manner. Example suitable techniques include, but are not limited to, press fit connections, adhesive connections, barbed connections, threaded connections, luer lock connections, bayonet connections, snap connections, and/or other suitable connections. Optionally, to facilitate a permanent seal, adhesive, molding techniques, etc. may be used with one or more other suitable coupling techniques. In one example coupling configuration, the first port 311a may be configured to couple with the first tubing subset 312a via a press fit, as in the configuration depicted in FIG. 5, where the first tubing subset 312a may be advanced toward and into the first port 311a, the inner tubing 312a′ may be advanced into the first opening 313a and into a water reservoir, and the end surface 322 of the outer tubing 312a″ may be placed in contact with a surface of the shoulder 316 facing the end surface 322 such that an outer surface of the outer tubing 312a″ may create a press fit connection with the first port 311a (e.g., with an inner surface or other suitable surface of first port 311a).

The second port 311b be configured to couple with a second manifold system or tubing subset 312b (e.g., a refill tubing subset or manifold system) having a first end configured to couple with the second port 311b and a second end configured to couple with a water reservoir in addition to the reservoir to which the fitting is coupled. The additional reservoir may have a smaller, the same size, or larger capacity than the water reservoir to which the fitting is coupled. In some cases, the additional reservoir may be a bottle, a bag, and/or other suitable reservoir configured to include water for use in an endoscope procedure. In addition to or as an alternative to the second port 311b being configured to couple to the second tubing subset 312b, the second port 311b may be configured to couple with other tubing in communication with an endoscope system including, but not limited to, irrigation tubing subsets, air/water tubing subsets, a connector in communication with multiple tubing subsets, and/or other suitable tubing or connectors thereof.

The second port 311b may include, among additional or alternative features, an elongated inner member 324, an elongated outer member 326, and a surface 328 defined between the inner member 324 and the outer member 326. In some cases, the inner member 324 may include a barb 330. The inner member 324, the outer member 326, and/or the barb 330, when included, may extend entirely or at least partially around a circumference of the opening or hole 313. The outer member 326 may include threads on an outer surface configured to receive a cap (not shown) that may be part of or separate from the second tubing subset 312b and configured to hermetically seal the second port 311b from atmosphere when the second port 311b is not in use (e.g., when the second port 311b is not coupled to the second tubing subset 312b). Although the second port 311b is depicted in FIG. 5 as including the inner member 324 and the outer member 326, the second port 311b may be configured with a single member and/or other suitable configuration configured to couple with the second tubing subset 312b.

The second tubing subset 312b may have one or more lumens configured to pass water from an additional water reservoir to a primary water reservoir to which a fitting is coupled (e.g., the cap 310). As depicted in FIG. 5, the second tubing subset 312b may include an inner tubing 312b′ at least partially defining the lumen through which water is to pass and which may have an outer diameter configured to fit within the hole or opening through the inner member 324. Further, a space 332 may be formed in an end of the second tubing subset 312b between an outer surface of the inner tubing 312b′ and an inner surface of the outer tubing 312b″ and the space 332 may be configured to receive a component of the second port 311b (e.g., the inner member 324 of the second port 311b).

The second port 311b may be configured to couple with the second tubing subset 312b and/or other suitable tubing or tubing subsets of the tubing set 312 in any suitable manner. Example suitable connection techniques include, but are not limited to, press fit connections, adhesive connections, barbed connections, threaded connections, luer lock connections, bayonet connections, snap connections, and/or other suitable connections. Optionally, to facilitate a permanent seal, adhesives, molding techniques, etc. may be used with one or more other suitable coupling techniques. In one example configuration, the second port 311b may be configured to couple with the second tubing subset 312b via a barbed press fit, as shown in FIG. 5. In such a configuration, the inner member 324 of the second port 311b may be configured to be inserted into the space 332 at the end of the second tubing subset 312b and the outer member 326 may be spaced from the inner member 324 a distance that facilitates receiving the outer tubing 312b″ between the inner member 324 and the outer member 326. When the second tubing subset 312b is received between the inner member 324 and the outer member 326, the barb 330 may engage an inner surface of the outer tubing 312b″, the outer member 326 may engage an outer surface of the outer tubing 312b″ and the surface 328 between the inner member 324 and the outer member 326 may engage an end surface 334 of the second tubing subset 312b to create a barbed press fit connection between the second tubing subset 312b and the second port 311b.

Although two configurations of the ports 311 of the fitting or cap 310 are described herein, other suitable configurations of the ports 311 are contemplated, including other configurations of the ports 311 configured to couple to different tubing subsets and/or refill containers. Further, although two ports 311 are described with respect to the cap 310, the cap 310 or the fitting may include any other suitable number of ports 311, as desired.

To facilitate having a single fitting or cap 310 for connecting to both a primary water reservoir and a supplemental water reservoir (e.g., a refill water reservoir), a second end (not shown) of the second or second tubing subset 312b may be configured to couple with a first port 311a of the supplemental water reservoir(s). Such a configuration of the refill or second tubing subset 312b and the ports 311 may facilitate coupling as many additional or supplemental water reservoirs to a primary water reservoir, as desired.

Further, in some cases, the second port 311b may be configured in a similar manner to ports on an endoscope umbilical. When so configured, a single tubing subset configuration may be utilized for coupling the first port 311a on a primary water reservoir to an endoscope umbilical and for coupling a first port 311a on a second supplemental water reservoir to the second port 311b of the primary water reservoir.

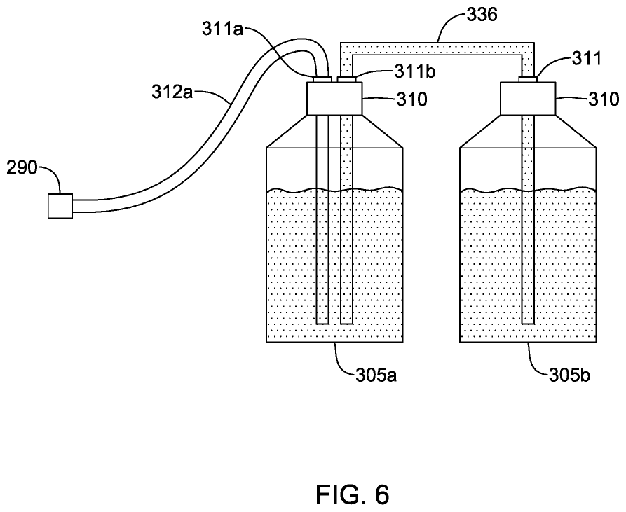

FIG. 6 schematically depicts an illustrative fluid supply system including a primary water reservoir 305a, a secondary water reservoir 305b, and a tubing set 312 having a first tubing subset 312a and a second tubing subset 312b, where the second tubing subset 312b may be configured as a bridge siphon 336 for fluidly coupling two or more water reservoirs to one another for use with an endoscope. The bridge siphon 336, as depicted in FIG. 6, may couple the primary water reservoir 305a to the supplemental or secondary water reservoir 305b. In one example, the primary water reservoir 305a may have a first port 311a coupled to a first end of the first tubing subset 312a (e.g., air/water tubing, irrigation tubing, etc.) having a second end with a connector 290 configured to connect with an endoscope umbilical and a second port 311b configured to connect to an end of the bridge siphon 336. In the example, the secondary water reservoir 305b may have a port 311 coupled to another end of the bridge siphon 336 such that the secondary water reservoir 305b and the primary water reservoir 305a are in fluid communication with one another via the bridge siphon 336.

In some cases, a plurality of bridge siphons 336 may be utilized, as desired, to fluidly couple one or more additional secondary water reservoirs 305b to the primary water reservoir 305a and an initial secondary water reservoir 305b, where the additional secondary water reservoirs 305b may include additional ports (e.g., in caps 310 and/or at other suitable locations) configured to receive an appropriate end of the additional bridge siphons 336. Additionally or alternatively, a single bridge siphon 336 may be configured to couple the primary water reservoir 305a to a plurality of secondary water reservoirs 305b.

The bridge siphon 336 may be formed from any suitable material. For example, the bridge siphon 336 may be formed from a rigid, semi-rigid, or flexible polymer, metal, alloy, mixture of polymers, mixture of polymers and metals, and/or other suitable materials.

In operation, once the bridge siphon 336 has been coupled to at least two water reservoirs (e.g., a primary water reservoir 305a and a secondary water reservoir 305b), the bridge siphon may be primed with water. Although other suitable techniques for priming the bridge siphon 336 are contemplated, one illustrative technique includes turning one or both of the primary and secondary water reservoirs 305a, 305b upside down to fill the bridge siphon 336 with water. The bridge siphon 336 depicted in FIG. 6 has been primed, such that water is in the bridge siphon 336. Once primed, the fluid supply system may supply water to an endoscope on-demand via the first tubing subset 312a from the primary water reservoir 305a and the secondary water reservoir 305b fluidly coupled to the primary water reservoir 305a via the second tubing set 312b (e.g., the bridge siphon 336).

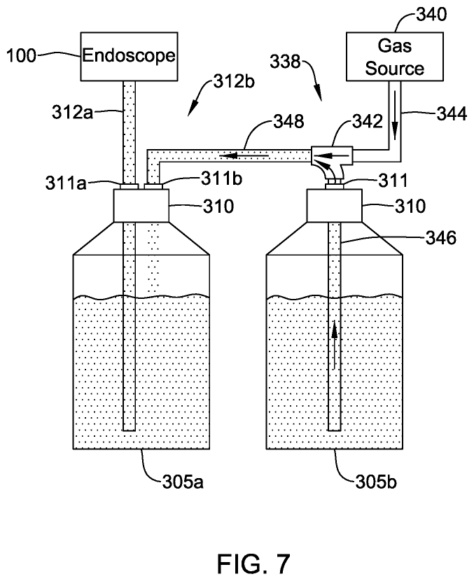

FIG. 7 schematically depicts an illustrative fluid supply system including a primary water reservoir 305a, a secondary water reservoir 305b, and a tubing set 312 having a first tubing subset 312a and a second tubing subset 312b, where the second tubing subset 312b may be configured as a venturi siphon 338 for fluidly coupling two or more water reservoirs 305 to one another for use with an endoscope. The venturi siphon 338, as depicted in FIG. 7, may couple the primary water reservoir 305a to the supplemental or secondary water reservoir 305b and may include or be fluidly coupled with a gas source 340 (e.g., air source, gas provided to a gas port of a connector 290, and/or other suitable gas source). In some cases, endoscope controls may be in electrical and/or mechanical communication with the gas source 340 so as to initiate a gas flow in response to a user of the endoscope calling for water, which may result in pressurizing the second tubing subset 312b and the primary and/or secondary water reservoirs 305a, 305b to facilitate providing water to the endoscope 100 (e.g., via an endoscope connector 290 or other suitable pathway) in response to the call for water.

The venturi siphon 338 may include a venturi connector 342 (e.g., a Y-connector or other suitable connector) connecting a pressurized flow tubing 344 (e.g., a length of the second tubing subset 312b) having a fluid path receiving a gas flow from the gas source 340, supplemental water tubing 346 (e.g., a length of the second tubing subset 312b) having a fluid path receiving a water flow from the supplemental or secondary water reservoir 305b, and a pressurized gas and water tubing 348 (e.g., a length of the second tubing subset 312b) receiving the gas flow and the supplemental water flow. In one example configuration of the fluid supply system utilizing a venturi siphon, the primary water reservoir 305a may have a first port 311a coupled to a first end of the first tubing subset 312a (e.g., air/water tubing, irrigation tubing, etc.) having a second end (not shown) configured to connect with the endoscope 100 (e.g., an endoscope umbilical of the endoscope 100) and a second port 311b configured to connect to the pressurized gas and water tubing 348. In the example, the secondary water reservoir 305b may have a port 311 coupled to the supplemental water tubing 346 such that the secondary water reservoir 305b and the primary water reservoir 305a are in fluid communication with one another via the venturi siphon 338.

In operation of a fluid supply system utilizing a venturi siphon, once a gas flow has been initiated from the gas source 340 through a pressurized flow tubing 344, the gas flows through the venturi connector 342 and into the primary water reservoir 305a and/or to atmosphere, and a pressure differential between the pressure in the venturi connector 342 and the pressure in the supplemental water tubing 346 created by the gas flow moving through the venturi connector 342 may cause water from the secondary water reservoir 305b to flow through the second tubing set 312b with the gas from the pressurized gas source to the primary water reservoir 305a. As such, as a user utilizes a lens wash feature or irrigation feature of the endoscope system 200, 300, for example, the primary water reservoir 305a may be automatically replenished with water and remain at a desired pressure.

The venturi connector 342 may be configured in any suitable manner. In some cases, the venturi connector 340 may be separate from and couple to or may be integrated in the fitting or cap 310 of the secondary water reservoir 305b. Further, the venturi connector 340 may include luer fittings or other suitable connecting features configured to engage tubing of the second tubing subset 312b leading to the gas source 340 and one or more secondary water reservoirs 305b. Alternatively or additionally, the venturi connector 340 may be integrated into one or more of the gas source 340, the primary water reservoir 305a, and the one or more secondary water reservoirs 305b.

In some cases, the venturi connector 342 may include one or more valves. In one example, a valve included in the venturi connector 342 may have a resting or biased position in which an outlet of the venturi connector 342 is blocked (e.g., the valve is in a closed position). In the example, when the venturi connector 342 is fluidly coupled with at least one secondary water reservoir 305b, the valve may be automatically or manually adjusted to an opened position to allow water to be drawn or pulled from the secondary water reservoir 305b in response to gas flowing through the venturi connector 342. Utilizing a valve in the venturi connector 342 may facilitate utilizing a second tubing subset 312b with both of 1) only a primary water reservoir 305a and 2) one or more secondary water reservoirs 305b.

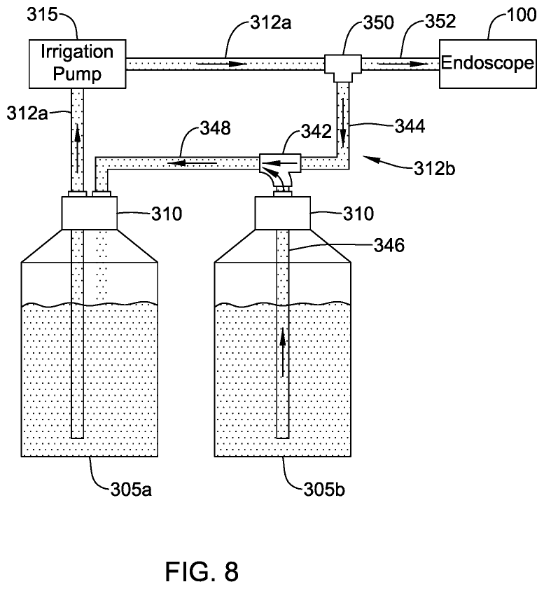

FIG. 8 schematically depicts an illustrative fluid supply system for a supply of irrigation fluid to the endoscope 100, which includes the venturi siphon 338 in a setup similar to that depicted in and discussed with respect to FIG. 7. However, the venturi siphon 338 depicted in FIG. 8 may be coupled to a water supply from the irrigation pump 315 via a valve 350 rather than to a gas source, as depicted in FIG. 7. In such a configuration, the fluid supply system may be set up to provide the primary water reservoir 305a with less than, the same as, or more than an amount of water than is removed from the primary water reservoir 305a during calls for irrigation. In one example, the fluid supply system may be set up to provide the primary water reservoir 305a with more than an amount of water that is removed from the primary water reservoir 305a during calls for irrigation due to water flowing both from the output of the irrigation pump and the secondary water reservoir 305b to the primary water reservoir 305a. The amount of fluid provided to the primary water reservoir 305a may be dependent on a pump speed or rate of the irrigation pump 315, diameters of tubing of the second tubing subset 312b, an amount of water proportioned to the venturi connector 342, and/or other suitable factors. In some cases, the amount of fluid provided to the primary water reservoir 305a may be configured to maintain a desired pressure in the primary water reservoirs 305a and/or a sufficient volume of water in the primary water reservoir 305a.

The venturi siphon 338 depicted in FIG. 8 may include or may be fluidly coupled to the valve 350, which may direct a first portion of a pressurized water flow outputted from the irrigation pump 315 to the endoscope 100 via irrigation tubing 352 (e.g., a length of the first tubing subset 312a, where another length of first tubing subset 312a may extend from the primary water reservoir 305a to the irrigation pump 315) and may direct a second portion of the pressurized water flow outputted from the irrigation pump 315 to the venturing connector 342 via the pressurized fluid tubing 344.

Although other types of valves are contemplated, the valve 350 may be a proportioning valve. In some cases, the proportioning valve may be adjustable such that a user may be able to adjust the proportion of the water outputted by the irrigation pump that is provided to the venturi connector 342, but this is not required and the proportioning valve may be fixed to as to provide a consistent non-adjustable flow of water to the endoscope 100 and the venturi connector 342.

In some cases, the pressurized flow tubing 344 may have a diameter that is smaller than a diameter of the first tubing subset 312a receiving fluid output from the irrigation pump 315. In such cases, the reduced diameter of the pressurized flow tubing 344 may result in increasing a velocity of fluid across the venturi connector 342 to create a desired pressure differential at the venturi connector 342 and draw water from the secondary water reservoir 305b.

In some cases, the venturi connector 342 and/or the proportioning valve 350 may include one or more valves configured to block fluid flow through the second tubing subset 312b when a secondary water reservoir 305b is not coupled to the second tubing subset 312b. In one example, a valve (e.g., a backflow preventer and/or other suitable valve) included in the venturi connector 342 and/or the proportioning valve 350 may have a resting or biased position in which an outlet of the venturi connector 342 and/or outlet of the proportioning valve 350 to the second tubing subset 312b is blocked (e.g., the valve is in a closed position). In the example, when the venturi connector 342 and/or the proportioning valve 350 is fluidly coupled with at least one secondary water reservoir 305b, the valve may be automatically or manually adjusted to an opened position to allow water to be drawn or pulled from the secondary water reservoir 305b in response to water flowing through the proportioning valve 350 and the venturi connector 342. Utilizing a valve in the venturi connector 342 and/or the proportioning valve 350 may facilitate utilizing a second tubing subset 312b with both of 1) only a primary water reservoir 305a and 2) one or more secondary water reservoirs 305b. Further, when utilizing the valve or backflow preventer in the proportioning valve 350 and/or the venturi connector 342, the proportioning valve 350 and/or the venturi connector 342 may be configured to directly couple to the secondary water reservoir 305b to allow the valve or backflow preventer to open (e.g., automatically upon connection or manually) and facilitate water flow from the second water reservoir 305b to the primary water reservoir 305a.

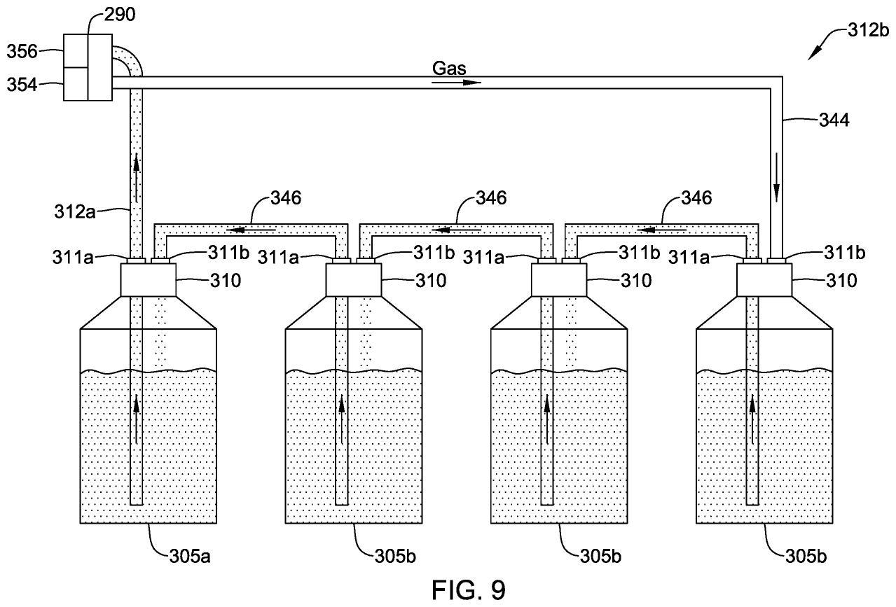

FIG. 9 schematically depicts an illustrative fluid supply system for an endoscope system 200, 300, which includes a primary water reservoir 305a, one or more secondary water reservoirs 305b, a first tubing subset 312a, and a second tubing subset 312b, where the primary water reservoir 305a and the one or more secondary water reservoirs 305b may be fluidly coupled to one another in a series via the second tubing subset 312b. The second tubing subset 312b depicted in FIG. 9 may include a pressurized flow tubing 344 extending from a gas port 354 of an endoscope umbilical connector 290 in fluid communication with a gas source of the endoscope system 200, 300 (e.g., via the umbilical of the endoscope system 20 and/or in one or more other suitable manners). The pressurized flow tubing 344 may be fluidly coupled to a second port 311b of the last secondary water reservoir 305b of one or more secondary water reservoirs 305b fluidly coupled to one another in series.

The second tubing subset 312b may further include supplemental water tubing coupling adjacent water reservoirs 305 to one another. As depicted in FIG. 9, supplemental water tubing 346 may extend from a first port 311a in one water reservoir 305 to a second port 311b in an additional water reservoir 305 and the water reservoirs 305 may be fluidly coupled to one another in this manner until the primary water reservoir 305a is reached. The water received at the primary water reservoir 305a may be received through the second port 311b and outputted via the first tubing subset 312a via the first port 311a in the primary water reservoir 305a.

In operation, the connector 290 may be coupled to an umbilical of the endoscope system 200, 300 and in response to a call for water at the endoscope 100, pressurized gas may be passed to a last secondary water reservoir 305b in a series of one or more secondary water reservoirs 305b. Adding the gas to the last secondary water reservoir 305b may pressurize the water reservoirs 305 with fluid (e.g., gas and/or liquid) and the second tubing subset 312b in response to water being removed from and provided to the endoscope 100 from the primary water reservoir 305a. As water is removed from a supplemental water reservoir 305b, the volume of the supplemental or secondary water reservoir 305b may be filled with water until the water is exhausted and then, filled with gas. As such, when water is removed from the primary water reservoir 305a, the water from the secondary water reservoirs 305b may be added to the primary water reservoir 305a to maintain a desired pressure and a water supply in the primary water reservoir 305a. In response to the water supply and pressure in the primary water reservoir 305a, fluid may be outputted through the first tubing subset 312a and the water port 356 of the connector 290.

Although three secondary water reservoirs 305b are depicted in FIG. 9, any suitable number of secondary water reservoirs 305b may be utilized. Alternatively or additionally, the fluid supply system depicted in FIG. 9 may be utilized with no secondary water reservoirs 305b and the pressurized flow tubing may be directly fluidly coupled to the second port 311b of the primary water reservoir 305a.

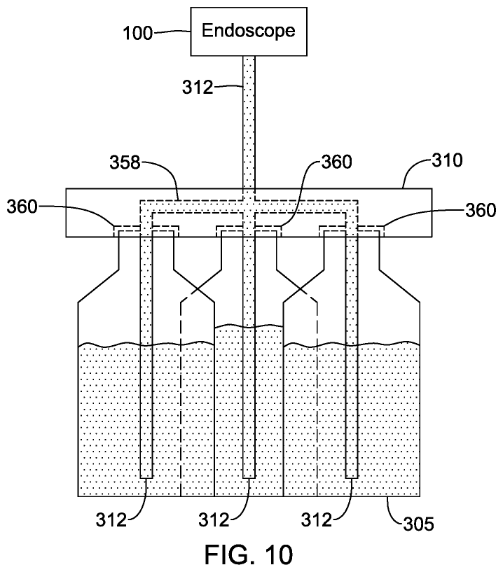

FIG. 10 schematically depicts an illustrative fluid supply system configured to supply water from a plurality of water reservoirs 305 to an endoscope 100, where a fitting or cap 310 may include a manifold system 358 in fluid communication with tubing 312 configured to extend into water reservoirs 305 connected to the fitting or cap 310. FIG. 10 depicts three water reservoirs 305 coupled to the fitting or cap 310, however, the fitting or cap 310 may be configured to connect to one, two, three, four, or more water reservoirs 305, as desired. Further, although FIG. 10 depicts the fitting or cap in fluid communication with the endoscope 100, the tubing set 312 depicted in FIG. 10 may be a second tubing subset 312b and the water reservoirs may be secondary water reservoirs 305b, such that the second tubing subset 312b may provide fluid from the secondary water reservoirs 305b coupled to the fitting or cap 310 to a primary water reservoir 305a fluidly coupled to the second tubing subset 312b and the endoscope 100.

When a user calls for fluid at the endoscope 100, the fluid in the water reservoirs 305 may be pressurized and fluid may be outputted to the endoscope via the tubing set 312. The fluid in the water reservoirs 305 may be pressurized in response to a pump acting on the fluid in the water reservoir 305 and/or the tubing set 312 (e.g., via an irrigation pump), a venturi siphon, a bridge siphon, a gas source and/or other suitable pressurizing technique. In one example, the tubing set 312 may be a dual lumen tubing set and gas may be inserted into the water reservoirs 305 via a first lumen of the dual lumen tubing set 312 and water may be outputted from the water reservoirs 305 via a second lumen of the dual lumen tubing set 312. Other suitable pressurizing configurations are contemplated.

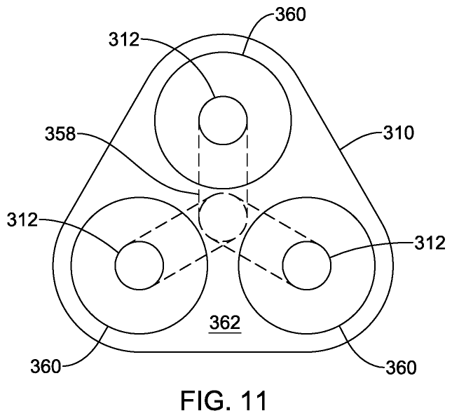

FIG. 11 schematically depicts a bottom view of the fitting or cap 310 depicted in FIG. 10. As depicted in FIG. 11, the fitting or cap 310 may include three connecting ports 360, or any other suitable number connecting ports 360, extending through a surface 362 of the fitting or cap 310. The tubing set(s) 312 may be in fluid communication with the manifold system 358 and extend through the surface 362 at each connecting port 360.

The fitting or cap 310 in the configurations of FIG. 10, FIG. 11, and/or other configurations discussed herein may be configured to connect with the water reservoirs 305 in any suitable manner. Example suitable connection types include, but are not limited to, threaded connections, snap connections, ring-detent connections, luer lock connections, bayonet connections, press fit connections, and/or other suitable types of connections. When the connection type is threaded and the fitting or cap 310 is configured to connect to multiple water reservoirs 305 via the connecting ports 360, as depicted in FIGS. 10 and 11, the water reservoirs 305 may be rotated to engage threads of the water reservoir 305 with stationary threads of the connecting ports 360 in the fitting or cap 310 (e.g., where the threads of the fitting or cap 310 may be molded into the structure of the fitting or cap 310 and/or otherwise are stationary with respect to other components of the fitting or cap 310).

Alternatively or additionally, the threads of the fitting or cap 310 may be individually adjustable such that threads of the fitting or cap 310 that are configured to connect to a water reservoir 305 may be individually rotated independent of other components of the fitting or cap 310. That is, a water reservoir 305 may be connected to the fitting or cap 310 without rotating the water reservoir 305 and/or threads or components of the fitting or cap 310 not intended to connect to threads of the water reservoir 305.

Various configurations of a fluid supply system have been described herein. It is contemplated that the various configurations, including various pressurization techniques and tubing sets or manifold systems, may be utilized with one another in combination and/or as alternatives. In one example, a tubing set or manifold system of a fluid supply system main include a venturi siphon fluidly coupling a plurality of secondary water reservoirs 305b and a bridge siphon for fluidly coupling one of the plurality of secondary water reservoirs 305b to a primary water reservoir 305a. Other suitable combinations and configurations of the fluid supply systems are contemplated.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A fluid supply system for an endoscope, the system comprising:
a reservoir defining a volume;
a first port in fluid communication with the volume and defining a first lumen and a first connector;
a second port in fluid communication with the volume and having a second lumen and a second connector;
a first tubing subset having a tube with a first end configured to couple to the first port and a second end configured to couple to an endoscope component; and
a second tubing subset having a tube with a first end configured to couple to the second port and a second end configured to couple to a first port in fluid communication with a volume of an additional reservoir.

2. The fluid supply system of claim 1, further comprising:
a cap configured to couple to the reservoir, and
wherein the cap includes the first port and the second port.

3. The fluid supply system of claim 1, wherein the first end of the tube of the first tubing subset is configured to be permanently secured to the first port.

4. The fluid supply system of claim 1, wherein:
the second end of the tube of the first tubing subset is configured to couple to an endoscope includes an endoscope connector having a water port and a gas port; and
the second end of the tube of the second tubing subset is configured to couple to the port in fluid communication with a volume of the additional reservoir includes an endoscope connector having a water port and a gas port.

5. The fluid supply system of claim 1, wherein the second tubing subset comprises a first fluid path in communication with a gas source and a second fluid path in communication with the volume of the additional reservoir.

6. The fluid supply system of claim 5, wherein the second tubing subset comprises a connector coupling a first length of tubing including the first fluid path and a second length of tubing including the second fluid path with a third length of tubing including the first fluid path and the second fluid path.

7. The fluid supply system of claim 1, wherein the first tubing subset is in fluid communication with a pump to pump fluid out of the volume of the reservoir to the endoscope.

8. The fluid supply system of claim 7, wherein:
the first tubing subset includes a first length of tubing extending from the reservoir to the pump and a second length of tubing from the pump to a valve;
the valve is in fluid communication with tubing configured to extend to the endoscope and the second tubing subset; and
the valve is configured to proportion a portion of the fluid received at the valve to the tubing configured to extend to the endoscope and a portion of the fluid received at the valve to the second tubing subset.

9. The fluid supply system of claim 8, wherein the second tubing subset comprises:
a connector;
a first length of tubing in fluid communication with the valve;
a second length of tubing configured to be in fluid communication with the volume of the additional reservoir; and
a third length of tubing in fluid communication with the reservoir and configured to receive fluid from the first length of tubing and the second length of tubing, and
wherein the connector couples the first length of tubing and the second length of tubing with the third length of tubing.

10. The fluid supply system of claim 1, wherein:
the reservoir is a first reservoir of a plurality of reservoirs and the plurality of reservoirs include a last reservoir;
the first tubing subset is configured to supply water from the first reservoir to a water port of an endoscope connector at the second end of the tube of the first tubing subset;
the second end of tube of the second tubing subset is configured to be in fluid communication with water from the last reservoir; and
a second tube of the second tubing subset having a first end configured to couple to a first port in communication with a volume of the last reservoir and a second end configured to be in fluid communication with a gas port on the endoscope connector.

11. A tubing set for use with an endoscope system, the tubing set comprising:
a first length of tubing having a first end and a second end;
an endoscope connector including a gas port and a water port, the water port is fluidly coupled to the first end of the first length of tubing; and
a manifold system configured to fluidly couple a plurality of reservoirs to provide water from the plurality of reservoirs to a water port of the endoscope connector.

12. The tubing set of claim 11, wherein the manifold system is incorporated in a cap configured to couple to the plurality of reservoirs and be in fluid communication with the second end of the first length of tubing.

13. The tubing set of claim 12, wherein the cap includes a plurality of sets of threads, each set of threads is configured to couple to a reservoir of the plurality of reservoirs.

14. The tubing set of claim 11, wherein the manifold system comprises:
a second length of tubing configured to fluidly couple a second reservoir to a first reservoir of the plurality of reservoirs, the first reservoir is in fluid communication with the second end of the first length of tubing;
a third length of tubing configured to fluidly couple a third reservoir of the plurality of reservoirs to the second reservoir; and
a fourth length of tubing coupled to the gas port of the endoscope connector and configured to fluidly couple the gas port to the third reservoir.

15. The tubing set of claim 11, wherein the manifold system comprises:
a second length of tubing configured to be in fluid communication with a first reservoir of the plurality of reservoirs;
a third length of tubing configured to be in fluid communication with a second reservoir of the plurality of reservoirs;
a fourth length of tubing configured to be in fluid communication with a fluid source; and
a connector coupling a second length of tubing, the third length of tubing, and the fourth length of tubing, the connector is configured to cause fluid from the second reservoir to travel through the third length of tubing to the second length of tubing in response to the manifold receiving fluid from the fourth length of tubing.

16. The tubing set of claim 15, wherein the fluid source is a gas source.

17. The tubing set of claim 15, wherein the fourth length of tubing is configured to fluidly couple to a gas port of the endoscope connector and the fluid source is a source of gas provided to the endoscope connector.

18. The tubing set of claim 15, wherein the fluid source is a water source.

19. A fluid supply system for an endoscope, the system comprising:
a plurality of water reservoirs;
a manifold system in communication with the plurality of water reservoirs; and
an endoscope connector configured to connect to an endoscope and in fluid communication with at least one of the plurality of water reservoirs, and
wherein the manifold system is configured to provide water from the plurality of reservoirs through the endoscope connector in response to a call for water from the endoscope.

20. The fluid system of claim 19, wherein a volume of the plurality of reservoirs and a fluid path through the manifold system are pressurized in response to the call for water from the endoscope.
